(19) United States
(12) Patent Application Publication
(10) Pub. No.: US 2016/0129199 A1
(43) Pub. Date: May 12, 2016

(54) Injection Device

(71) Applicant: Cilag GmbH International, Zug (CH)

(72) Inventors: Ahmad Bitar, Cambridgeshire (GB); Douglas Ivan Jennings, Hertfordshire (GB)

(21) Appl. No.: 14/897,347

(22) PCT Filed: Jun. 11, 2014
(86) PCT No.: PCT/EP2014/062163

§ 371 Date: Dec. 10, 2015

(30) Foreign Application Priority Data
Jun. 11, 2013 (GB) 1310394.0

(51) Int. Cl. A61M 5/32

(52) U.S. Cl. CPC A61M 5/3204

(57) ABSTRACT

An injection device comprising a housing adapted to receive a syringe having a discharge nozzle, and a sliding component which protrudes, when in a first position, from the aperture in the housing, wherein the sliding component is movable into the housing into a second position, wherein the sliding component comprises a contact surface located at a first end of the sliding component which protrudes from the housing when the sliding component is in its first position.

### FIELD OF THE INVENTION

The invention relates to an injection device of the type that receives a syringe, extends the syringe and discharges its contents, commonly known as an auto-injector.

### BACKGROUND OF THE INVENTION

Auto-injectors are known from WO 95/35126 and EP-A-0 516 473 and tend to employ a drive spring and some form of release mechanism that releases the syringe from the influence of the drive spring once its contents are supposed to have been discharged, to allow it to be retracted by a return spring.

An auto-injector is known from WO 2007/036676 which has a locking mechanism which must be disengaged before the release mechanism can be activated. In its locked position, the locking mechanism also prevents forward movement of the syringe out of the injection device against the bias of the return spring, for example when a cap gripping a boot covering the syringe needle, is removed. In the injection device described in WO 2007/036676, the locking mechanism comprises a sleeve which protrudes from an open end of the injection device. The sleeve is biased into its extended position by a resilient spring mechanism which must be overcome to disengage the locking mechanism. The locking mechanism can be disengaged by, for example, moving the sliding sleeve inwardly into the injection device. This can be done by forcing the end of the sliding sleeve against tissue and then activating the release mechanism.

Having the locking mechanism freely disengaged is undesirable because the release mechanism can be activated unintentionally causing accidental activation of the injection device. This is both dangerous and wasteful. Moreover, sleeves of conventional auto-injectors can be uncomfortable against the skin of a patent and are often unreliable in disengaging a locking mechanism to actuate the device.

### SUMMARY OF THE INVENTION

In accordance with the present invention, there is provided an injection device comprising:
a housing adapted to receive a syringe having a discharge nozzle, the syringe being moveable in the housing on actuation of the injection device along a longitudinal axis from a retracted position in which the discharge nozzle is contained within the housing and an extended position in which the discharge nozzle of the syringe extends from the housing through an exit aperture;
a sliding component which protrudes, when in a first position, from the aperture in the housing,
wherein the sliding component is movable into the housing into a second position,
wherein the sliding component comprises a contact surface located at a first end of the sliding component which protrudes from the housing when the sliding component is in its first position.

The sliding component may be a sliding sleeve, the purpose of which is to prevent accidental actuation of the device, as described further below.

The device may further comprise:
an actuator; and
a drive adapted to be acted upon by the actuator and in turn act upon the syringe to advance it from its retracted position to its extended position and discharge its contents through the discharge nozzle.

The sliding component may be part of a locking mechanism moveable from an engaged position in a direction into the housing at the exit aperture into a disengaged position and adapted to prevent actuation of the device when it is in its engaged position and permit actuation of the device when it is in its disengaged position.

By providing a contact surface, for example in the form of a flange, the locking mechanism can be more easily engaged and disengaged. This is because the contact surface provides an improved contact area against tissue. This means that point pressure from the locking mechanism applied to tissue is reduced. Moreover, the contact surface prevents the locking mechanism becoming caught, by friction or snagging, on the rim of the exit aperture. Thus, safer use of the injection device is achieved. The contact surface also makes the sliding component more visible to a user and provides improved and more reliable tactile feedback during operation of the device. The improved visibility and tactile feedback make it easier for a user to determine whether the sliding component has been moved fully into the housing to disengage the release mechanism. Thus, actuation of the device is more consistent, reliable and user-friendly and actuation failure is less likely. The visibility of the sliding component to a user can be further enhanced by providing a sliding component which is coloured differently to the housing, for example a brightly-coloured sliding component. Providing a contact surface, for example in the form of a flange, also limits the movement of the sliding component into the housing. The contact surface provides a position stop to prevent the sliding component being pushed too far into the housing. This prevents damage to the device, increases the user-friendliness of the device, and prevents injury to a user.

The contact surface may extend perpendicularly to a longitudinal axis of the sliding component. In other words, the contact surface may extend radially from the sliding component.

The first end of the sliding component may flare outwardly towards the contact surface. The sliding component may flare uniformly outwardly towards the contact surface. An advantage arising from a sliding component which flares outwardly towards a contact surface, such as a flange, at its first end, is that the risk of the skin of a patient being trapped between the flange and the housing when the sliding component is moved into the housing is significantly reduced. This is advantageous because the trapping of skin could cause pain to the patient and could result in a sudden movement resulting in further injury.

The contact surface may extend outwardly from the sliding component. For example, the contact surface may be located on an exterior surface of the first end of the sliding component.

Alternatively, the contact surface may extend inwardly from the sliding component. For example, the contact surface may be located on an interior surface of the first end of the sliding component. A configuration in which the contact surface extends inwardly from the sliding component is advantageous since it prevents the skin of a patient being trapped between the contact surface and the housing when the sliding component is moved into the housing, which could cause pain to the patient and could result in a sudden movement resulting in further injury.

The device may further comprise a cap removably located over the exit aperture. The cap may comprise a body and a sleeve located within the body and fixed relative to the body. An interior of the cap may be shaped to receive the contact surface. For example, the interior of the cap may comprise at least one rib or a plurality of ribs. The at least one rib or plurality of ribs may permit the cap to shapingly conform with the contact surface when the cap is over the exit aperture. In other words, the at least one rib or plurality of ribs may be configured such that the shape of the cap conforms with the contact surface of the sliding component so as to leave no gap, or minimal gap, between the ribs and the contact surface. The device may further comprise a retaining means on the housing and/or the cap for retaining the cap over the exit aperture. Providing a cap which shapingly conforms with the contact surface and which can be retained securely on the housing helps to protect the first end of the sliding component and, in particular, the contact surface so as to minimise the risk of damage or contamination to the device.

A first end of the housing closest to the contact surface may be shaped to receive the contact surface when the sliding component is moved in a direction into the housing. Therefore, the housing is adapted to receive the contact surface.

The contact surface may be removable from the sliding component. This is advantageous if it is desired to use an injection device with one or more different kinds of contact surface, or to retrofit a contact surface onto an existing injection device.

In any embodiment, the injection device may contain a substance selected from the group consisting of: golimumab, hormones, antitoxins, substances for the control of pain, substances for the control of thrombosis, substances for the control or elimination of infection, peptides, proteins, human insulin or a human insulin analogue or derivative, polysaccharide, DNA, RNA, enzymes, antibodies, oligonucleotide, antiallergics, antihistamines, anti-inflammatories, corticosteroids, disease modifying anti-rheumatic drugs, erythropoietin, or vaccines, for use in the treatment or prevention of rheumatoid arthritis, psoriatic arthritis, ankylosing spondylitis, ulcerative colitis, hormone deficiency, toxicity, pain, thrombosis, infection, diabetes mellitus, diabetic retinopathy, acute coronary syndrome, angina, myocardial infarction, atherosclerosis, cancer, macular degeneration, allergy, hay fever, inflammation, anaemia, or myelodysplasia, or in the expression of protective immunity.

By ‘the injection device may contain a substance’ it is meant that the substance may be contained within a suitable medicament container, such as a vial or syringe, within the injection device. Such medicament container may contain other substances, such as further active or inactive ingredients.

In a further aspect of the invention, a substance is provided, the substance being selected from the group consisting of: golimumab, hormones, antitoxins, substances for the control of pain, substances for the control of thrombosis, substances for the control or elimination of infection, peptides, proteins, human insulin or a human insulin analogue or derivative, polysaccharide, DNA, RNA, enzymes, antibodies, oligonucleotide, antiallergics, antihistamines, anti-inflammatories, corticosteroids, disease modifying anti-rheumatic drugs, erythropoietin, or vaccines, for use in the treatment or prevention of rheumatoid arthritis, psoriatic arthritis, ankylosing spondylitis, ulcerative colitis, hormone deficiency, toxicity, pain, thrombosis, infection, diabetes mellitus, diabetic retinopathy, acute coronary syndrome, angina, myocardial infarction, atherosclerosis, cancer, macular degeneration, allergy, hay fever, inflammation, anaemia, or myelodysplasia, or in the expression of protective immunity, by delivery of said substance to a human subject using an injection device according to any of the above embodiments.

In yet another aspect of the invention, an injection device is provided for use in the treatment or prevention of rheumatoid arthritis, psoriatic arthritis, ankylosing spondylitis, ulcerative colitis, hormone deficiency, toxicity, pain, thrombosis, infection, diabetes mellitus, diabetic retinopathy, acute coronary syndrome, angina, myocardial infarction, atherosclerosis, cancer, macular degeneration, allergy, hay fever, inflammation, anaemia, or myelodysplasia, or in the expression of protective immunity, by delivery of a substance selected from the group consisting of: golimumab, hormones, antitoxins, substances for the control of pain, substances for the control of thrombosis, substances for the control or elimination of infection, peptides, proteins, human insulin or a human insulin analogue or derivative, polysaccharide, DNA, RNA, enzymes, antibodies, oligonucleotide, antiallergics, antihistamines, anti-inflammatories, corticosteroids, disease modifying anti-rheumatic drugs, erythropoietin, or vaccines, to a human subject by using the injection device, where the injection device is an injection device of any of the above embodiments.

By ‘delivery of a substance’ it is meant that the injection device is used to inject said substance into the human subject, for example by subcutaneous, intradermal or intramuscular injection. Said substance may be administered in combination with other substances, such as further active or inactive ingredients.

### BRIEF DESCRIPTION OF THE DRAWINGS

The present invention is now described by way of example with reference to the accompanying drawings, in which:

FIG. 1 is a perspective end view of one end of injection device according to one embodiment of the invention before a cap is affixed to it;

FIG. 2 is a perspective end view of the injection device according to FIG. 1 once the cap has been affixed;

FIG. 3 is a side cross-sectional view of the injection device of FIG. 1;

FIGS. 4a and 4b are top cross-sectional views of the injection device of FIG. 1;

FIG. 5 is an enlarged cut-out from FIG. 4b;

FIG. 6 is a sectional schematic how an injection device may be further modified; and

FIG. 7 is a cut-away view of such a modified injection device;

FIGS. 8a and 8b show an end of an injection device according to an alternative embodiment of the invention;

FIG. 9 is a perspective view of an end of an injection device according to an embodiment of the invention showing a sliding component having a contact surface which extends inwardly;

FIG. 10 is a perspective view of an end of an injection device according to an embodiment of the invention showing a sliding component which flares towards a contact surface; and

FIG. 11 is a perspective view of a cap for an injection device according to an embodiment of the invention.

### DETAILED DESCRIPTION OF THE DRAWINGS

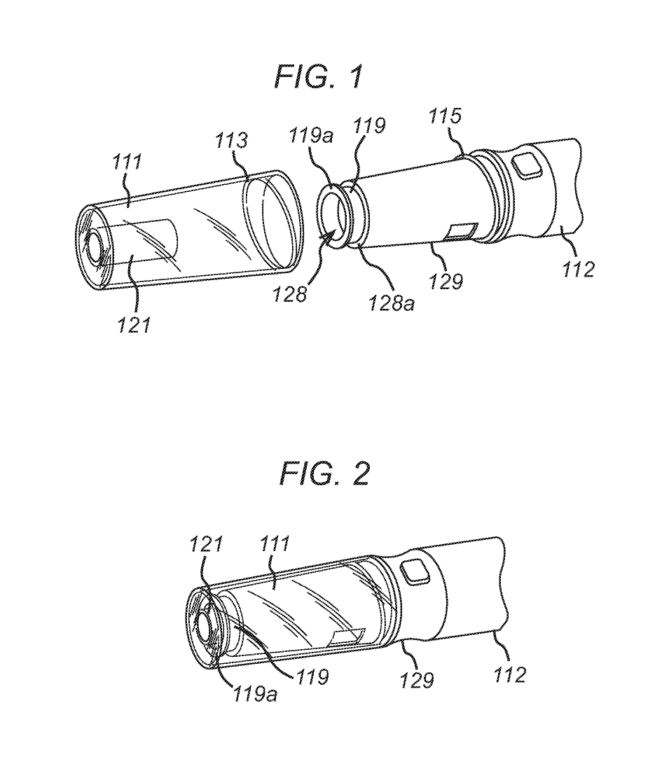

FIG. 1 shows the end of an injection device housing 112 and a cap 111. Other parts of the device will be described in greater detail below, but it will be seen that the cap 111 includes a thread 113 that cooperates with a corresponding thread 115 on the end of the housing. The end of the housing 112 has a case nose 129 and an exit aperture 128, from which the end of a sleeve 119 can be seen to emerge. The cap 111 has a central boss 121 that fits within the sleeve 119 when the cap 111 is installed on the housing 112, as can be seen in FIG. 2.

The sliding component 119 can take various forms. FIGS. 1 to 3 show a sliding component 119 which is a sliding sleeve. The sliding sleeve 119 comprises a contact surface 119a located at a first end of the sliding component 119 which protrudes from the housing 112 when the sliding component is in its first position. In the embodiment shown in FIGS. 1 to 3, the contact surface 119a comprises a flange which extends perpendicularly to a longitudinal axis of the sliding sleeve 119. In other words, the contact surface extends radially from the sliding component. In the embodiment shown in FIGS. 1 to 3, the flange 119a extends outwardly from the end of the sliding sleeve 119 and is located on an exterior surface of the first end of the sliding sleeve 119.

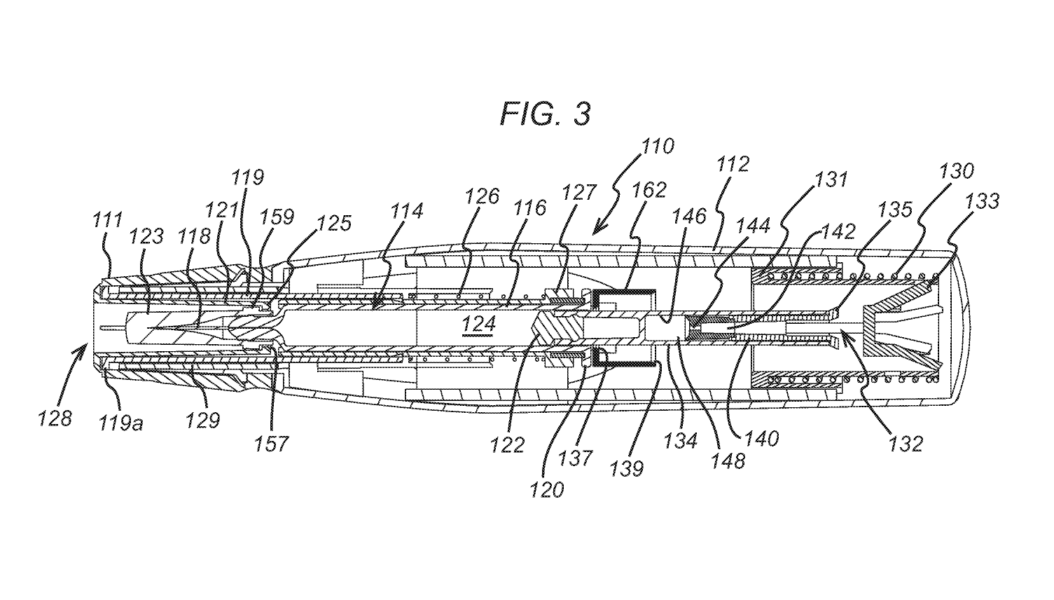

FIG. 3 shows an injection device 110 in more detail. The housing 112 contains a hypodermic syringe 114 of conventional type, including a syringe body 116 terminating at one end in a hypodermic needle 118 and at the other in a flange 120. The conventional plunger that would normally be used to discharge the contents of the syringe 114 manually has been removed and replaced with a drive element 134 that terminates in a bung 122. The bung 122 constrains a drug 124 to be administered within the syringe body 116. Whilst the syringe illustrated is of hypodermic type, this need not necessarily be so. Transcutaneous or ballistic dermal and subcutaneous syringes may also be used with the injection device of the present invention. As illustrated, the housing includes a return spring 126 that biases the syringe 114 from an extended position in which the needle 118 extends from an aperture 128 in the housing 112 to a retracted position in which the discharge nozzle 118 is contained within the housing 112. A case nose 129 is located at one end of the housing. The return spring 126 acts on the syringe 114 via a syringe carrier 127.

At the other end of the housing is an actuator, which here takes the form of a compression drive spring 130. Drive from the drive spring 130 is transmitted via a multi-component drive to the syringe 114 to advance it from its retracted position to its extended position and discharge its contents through the needle 118. The drive accomplishes this task by acting directly on the drug 124 and the syringe 114. Hydrostatic forces acting through the drug 124 and, to a lesser extent, static friction between the bung 122 and the syringe body 116 initially ensure that they advance together, until the return spring 126 bottoms out or the syringe body 116 meets some other obstruction (not shown) that retards its motion.

The multi-component drive between the drive spring 130 and the syringe 114 consists of three principal components. A drive sleeve 131 takes drive from the drive spring 130 and transmits it to flexible latch arms 133 on a first drive element 132. This in turn transmits drive via flexible latch arms 135 to a second drive element, the drive element 134 already mentioned.

The first drive element 132 includes a hollow stem 140, the inner cavity of which forms a collection chamber 142 in communication with a vent 144 that extends from the collection chamber through the end of the stem 140. The second drive element 134 includes a blind bore 146 that is open at one end to receive the stem 140 and closed at the other. As can be seen, the bore 146 and the stem 140 defining a fluid reservoir 148, within which a damping fluid is contained.

A trigger (not shown) is provided that, when operated, serves to decouple the drive sleeve 131 from the housing 112, allowing it to move relative to the housing 112 under the influence of the drive spring 130. The operation of the device is then as follows.

Initially, the drive spring 130 moves the drive sleeve 131, the drive sleeve 131 moves the first drive element 32 and the first drive element 132 moves the second drive element 134, in each case by acting through the flexible latch arms 133, 135. The second drive element 134 moves and, by virtue of static friction and hydrostatic forces acting through the drug 124 to be administered, moves the syringe body 116 against the action of the return spring 126. The return spring 126 compresses and the hypodermic needle 118 emerges from the exit aperture 128 of the housing 112. This continues until the return spring 126 bottoms out or the syringe body 116 meets some other obstruction (not shown) that retards its motion. Because the static friction between the second drive element 134 and the syringe body 116 and the hydrostatic forces acting through the drug 124 to be administered are not sufficient to resist the full drive force developed by the drive spring 130, at this point the second drive element 134 begins to move within the syringe body 116 and the drug 124 begins to be discharged. Dynamic friction between the second drive element 134 and the syringe body 116 and hydrostatic forces acting through the drug 124 to be administered are, however, sufficient to retain the return spring 126 in its compressed state, so the hypodermic needle 118 remains extended.

Before the second drive element 134 reaches the end of its travel within the syringe body 116, so before the contents of the syringe have fully discharged, the flexible latch arms 135 linking the first and second drive elements 132, 134 reach a constriction 137 within the housing 112. The constriction 137 moves the flexible latch arms 135 inwards from the position shown to a position at which they no longer couple the first drive element 132 to the second drive element 134, aided by the bevelled surfaces on the constriction 137. Once this happens, the first drive element 132 acts no longer on the second drive element 134, allowing the first drive element 132 to move relative to the second drive element 134.

Because the damping fluid is contained within a reservoir 148 defined between the end of the first drive element 132 and the blind bore 146 in the second drive element 134, the volume of the reservoir 146 will tend to decrease as the first drive element 132 moves relative to the second drive element 134 when the former is acted upon by the drive spring 130. As the reservoir 148 collapses, damping fluid is forced through the vent 144 into the collection chamber 142. Thus, once the flexible latch arms 135 have been released, the force exerted by the drive spring 130 does work on the damping fluid, causing it to flow though the constriction formed by the vent 144, and also acts hydrostatically through the fluid and through friction between the first and second drive elements 132, 134, thence via the second drive element 134. Losses associated with the flow of the damping fluid do not attenuate the force acting on the body of the syringe to a great extent. Thus, the return spring 126 remains compressed and the hypodermic needle remains extended.

After a time, the second drive element 134 completes its travel within the syringe body 116 and can go no further. At this point, the contents of the syringe 114 are completely discharged and the force exerted by the drive spring 130 acts to retain the second drive element 134 in its terminal position and to continue to cause the damping fluid to flow though the vent 144, allowing the first drive element 132 to continue its movement.

Before the reservoir 148 of fluid is exhausted, the flexible latch arms 133 linking the drive sleeve 131 with the first drive element 132 reach another constriction 139 within the housing 112. The constriction 139 moves the flexible latch arms 133 inwards from the position shown to a position at which they no longer couple the drive sleeve 131 to the first drive element 132, aided by the bevelled surfaces on the constriction 139. Once this happens, the drive sleeve 131 acts no longer on the first drive element 132, allowing them to move relative each other. At this point, of course, the syringe 114 is released, because the forces developed by the drive spring 130 are no longer being transmitted to the syringe 114, and the only force acting on the syringe will be the return force from the return spring 126. Thus, the syringe 114 is now returned to its retracted position and the injection cycle is complete.

All this takes place, of course, only once the cap 111 has been removed from the end of the housing 112. As can be seen from FIG. 3, the end of the syringe is sealed with a boot 123. The central boss 121 of the cap that fits within the sleeve 119 when the cap 111 is installed on the housing 112, is hollow at the end and the lip 125 of the hollow end is bevelled on its leading edge 157, but not its trailing edge. Thus, as the cap 111 is installed, the leading edge 157 of the lip 125 rides over a shoulder 159 on the boot 123. However, as the cap 111 is removed, the trailing edge of the lip 125 will not ride over the shoulder 159, which means that the boot 123 is pulled off the syringe 114 as the cap 111 is removed.

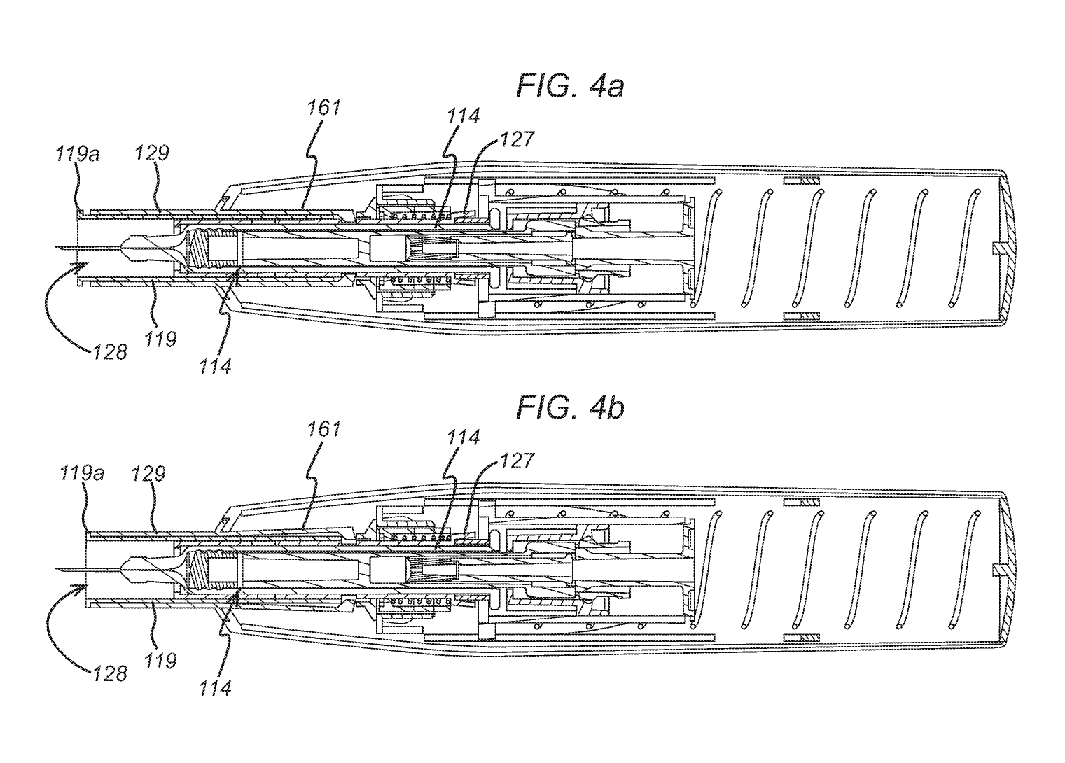

Meanwhile, as can best be seen in FIGS. 4 and 5, the syringe carrier 127, with respect to which the syringe 114 cannot move, is prevented from movement by a resilient latch member 161 that is located within the housing 112 and is biased into a position in which it engages a locking surface 163 of a syringe carrier 127. Before engaging the locking surface 163, the latch member 161 also extends thorough a latch opening 165 in the sleeve 119, the end of which projects from the exit aperture 128. The latch member 161 includes a ramped surface 167 against which an edge 171 of the latch opening 165 acts in the manner of a cam acting on a cam follower. Thus, movement of the sleeve 119 in a direction into the housing 112, or in other words depression of the projecting end of the sleeve, brings the edge 171 of the latch opening 165 into contact with the ramped surface 167 of the latch member 161 and further depression causes the latch member 161 to move outwards and thus to disengage from the locking surface 163. The sleeve 119 may be depressed by bringing the end of the injection device into contact with the skin at an injection site. Once the latch member 161 has disengaged from the locking surface 163, the syringe carrier 127 is free to move as required under the influence of the actuator and drive.

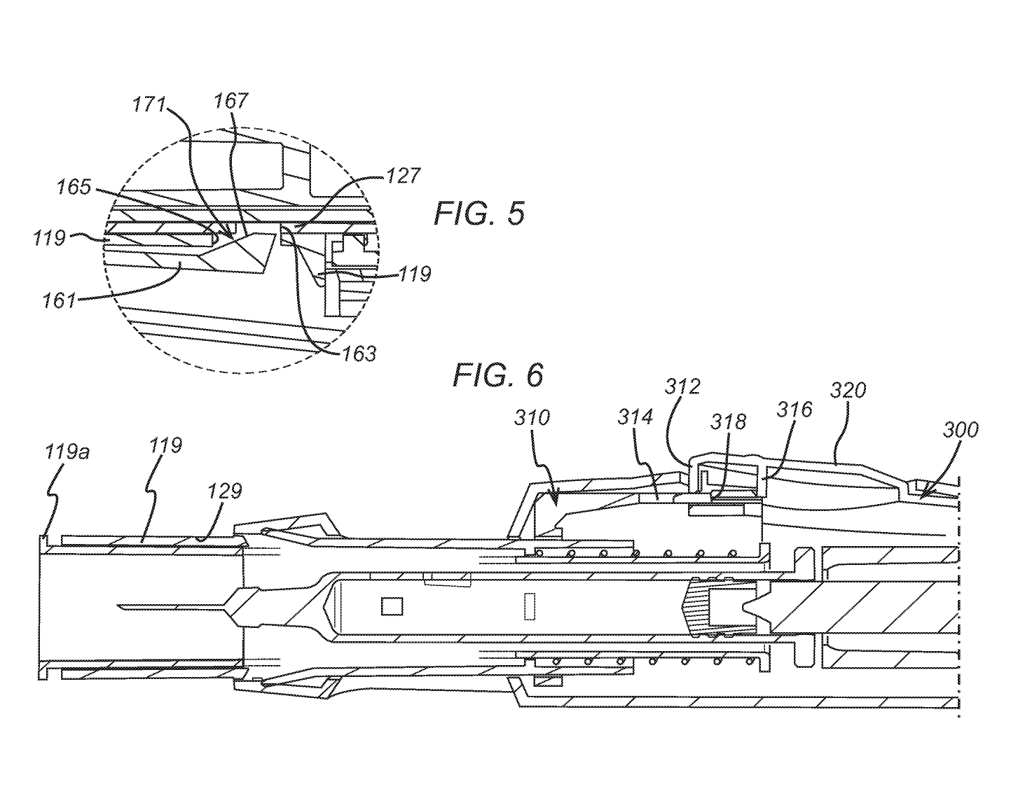

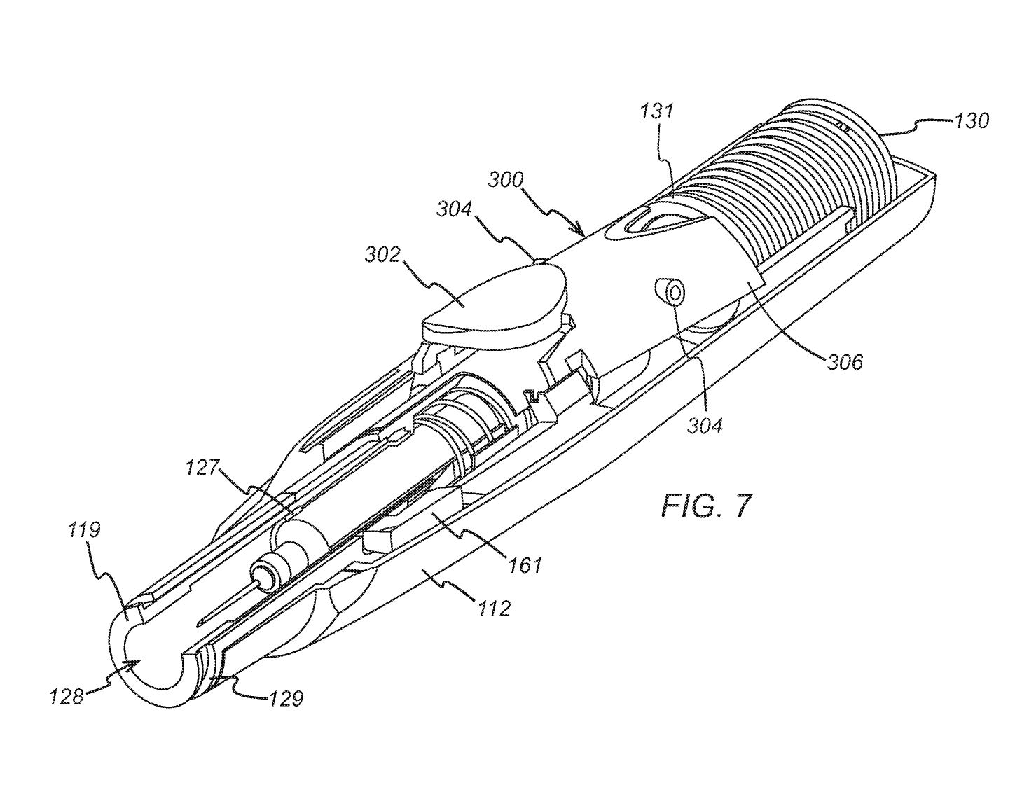

FIGS. 6 and 7 show how the device may be further modified. Although FIGS. 6 and 7 differ from FIGS.

4 and 5 in some details, the principles now discussed are applicable to the device shown in FIGS. 4 and 5. As can be seen, the device includes a trigger 300 having a button 302 at one end and a pair of lugs 304 that cooperate with pins (not shown) on the inside of the housing 112 to allow the trigger to pivot about an axis through the two lugs 304. The main body portion of the trigger 300, to which both the button 302 and the lugs 304 are affixed, forms a locking member 306. In the position shown, the end of the locking member 306 remote from the button 302 engages the end of the drive sleeve 131, against which the drive spring 130 acts and which in turn acts upon the multi-component drive previously discussed. This prevents the drive sleeve 131 from moving under the influence of the drive spring 130. When the button 302 is depressed, the trigger 300 pivots about the lugs 304, which lifts the end of the locking member 306 from its engagement with the drive sleeve 131, now allowing the drive sleeve 131 to move under the influence of the drive spring 130.

FIG. 7 shows the exit aperture 128 in the end of the housing 112, from which the end of the sleeve 119 can again be seen to emerge. As is shown in FIG. 6, the sleeve 119 is coupled to a button lock 310 which moves together with the sleeve 119. The trigger includes a stop pin 312 and the button lock 310 includes a stop aperture 314 which, as shown in FIG. 6, are out of register. They can, however, be brought into register by inward movement of the sleeve 119, which results in a corresponding movement of the button lock 310. Whilst the stop pin 312 and the stop aperture 314 are out of register, the button 302 may not be depressed; once they are in register, it may. The trigger 300 also includes a flexible, barbed latching projection 316 and the button lock 310 also includes a latching surface 318 with which the latching projection 316 engages when the button is depressed. Once the latching projection 316 has latched with the latching surface 318, the trigger 300 is permanently retained with the button 302 in its depressed position.

Thus, movement of the sleeve 119 in a direction into the housing 112, or in other words depression of the projecting end of the sleeve, brings the stop pin 312 into register with the stop aperture 314, allowing the trigger button 302 to be depressed, whereupon it is retained in its depressed position by the latching projection 316 and the latching surface 318. The sleeve 119 may be depressed by bringing the end of the injection device into contact with the skin at an injection site which, apart from anything else, ensures it is properly positioned before the injection cycle begins.

The use of the sleeve 119 both to release and lock the trigger 300 and to allow the syringe carrier 127 to move, together with a boot-removing cap 111 that prevents the sleeve 119 from being depressed results in an integrated injection device of elegant design.

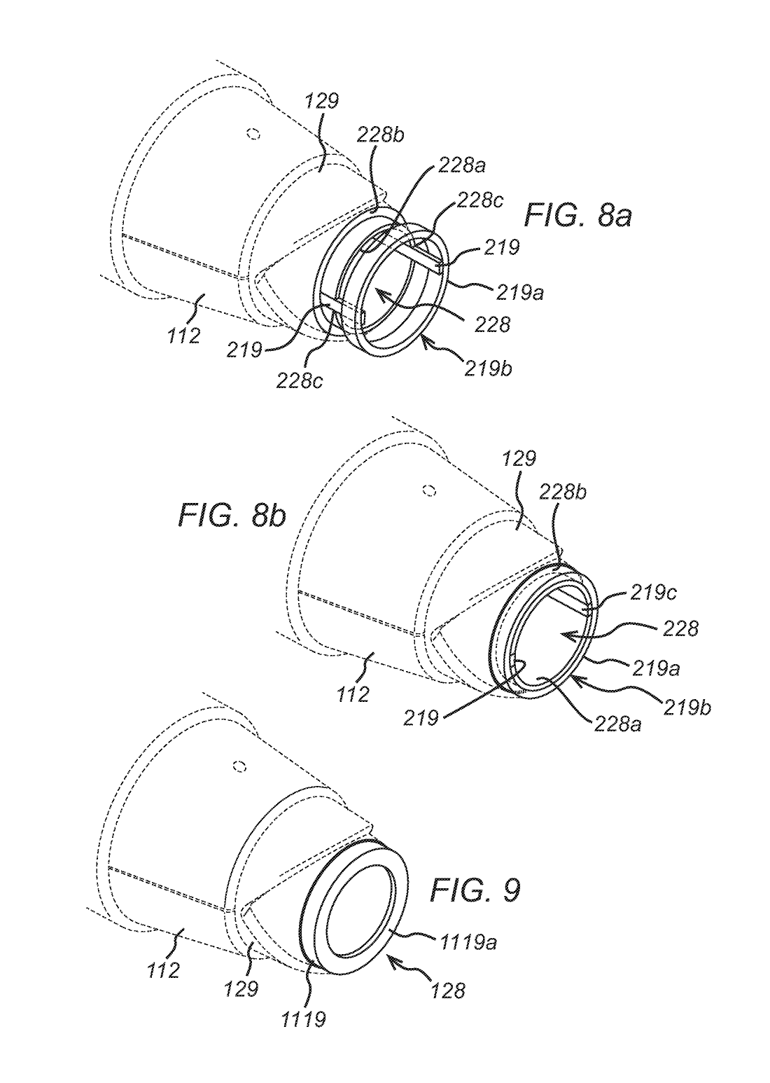

FIG. 8 shows an alternative embodiment of the end of the injection device 110. In exactly the same ways as discussed in connection with FIG. 1, the end of the housing 112 has an exit aperture 228 formed by rim 228a. Arms 219 which form part of the locking mechanism in exactly the same way as the sleeve 119 in FIGS. 1 to 5 emerge from the exit aperture 228. Each arm 219 is connected to a cylindrical end section 219a having an aperture. Each arm 219 is connected on the inside of the aperture. In a similar way to the flange 119a, the cylindrical end section 219a has a contact surface 219b which can contact tissue when pressed against it. The arms 219 sit and slide in slots 228c which extend through the end of the rim 228a. A shelf 228b on the housing extends around the circumference of the rim 228a and is adapted to receive the cylindrical end section 219a and prevent rearwards movement.

The cylindrical end section 219a can slide from a locked position in which the cylindrical end section 219a is spaced from the shelf 228b, to an unlocked position in which the cylindrical end section 219a has been pushed into a position in which it sits adjacent, in contacting juxtaposition, to the shelf 228b around the outside of the rim 228a. In all other aspects, the injection device 110 and locking mechanism operates in the same way as the sleeve 119 explained in connection with FIGS. 4a, 4b and 5 above.

As described above, the sliding component comprises a contact surface located at a first end of the sliding component which protrudes from the housing. By providing a contact surface, for example in the form of a flange, the locking mechanism of the injection device can be more easily engaged and disengaged. This is because the contact surface provides an improved contact area against tissue. This means that point pressure from the sliding component applied to tissue is reduced. Moreover, the contact surface prevents the sliding component becoming caught, by friction or snagging, on the rim of the exit aperture. Thus, safer use of the injection device is achieved. The contact surface also makes the sliding component more visible to a user and provides improved and more reliable tactile feedback during operation of the device. The improved visibility and tactile feedback make it easier for a user to determine whether the sliding component has been moved fully into the housing to disengage the locking mechanism. Thus, actuation of the device is more consistent, reliable and user-friendly and actuation failure is less likely. The visibility of the sliding component to a user can be further enhanced by providing a sliding component which is coloured differently to the housing, for example a brightly-coloured sliding component. Providing a contact surface, for example in the form of a flange, also limits the movement of the sliding component into the housing. The contact surface provides a position stop to prevent the sliding component being pushed too far into the housing. This prevents damage to the device, increases the user-friendliness of the device, and prevents injury to a user.

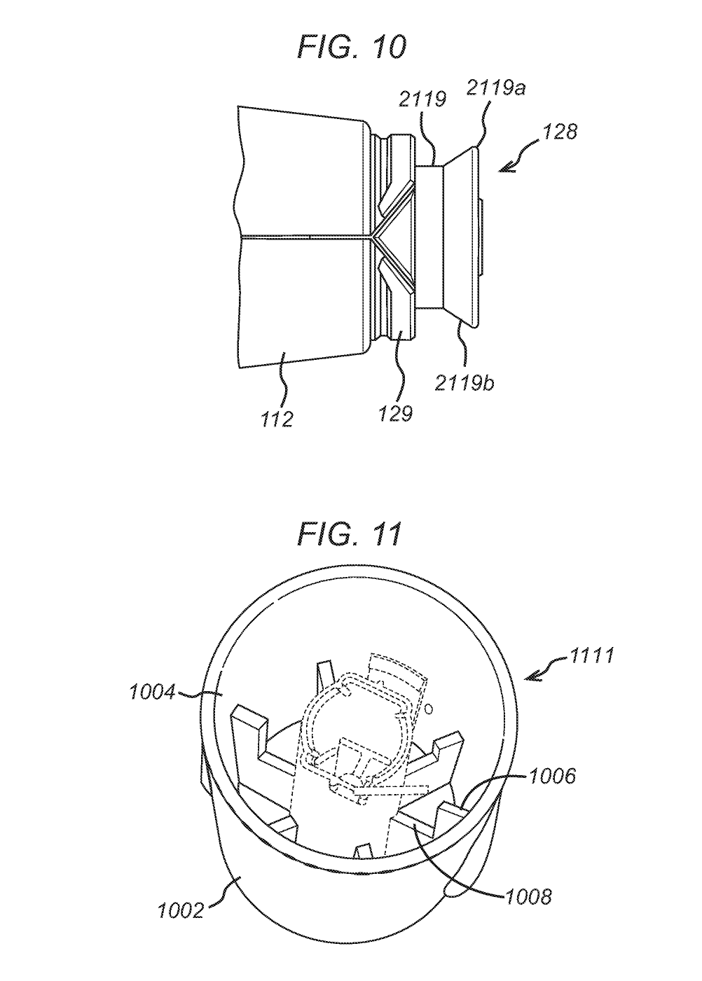

FIGS. 9 and 10 show examples of sliding components according to the invention. It will be understood by a person skilled in the art that any of these sliding components, as well as other alternatives, can be used with the injection device shown in FIGS. 1 to 8.

In the embodiment shown in FIG. 9, a flange 1119a extends inwardly from an end of a sliding component 1119 and is located on an interior surface of a first end of the sliding component 1119. This configuration is advantageous since it prevents the skin of a patient being trapped between the flange 1119a and the housing 112 when the sliding component 1119 is moved into the housing 112, which could cause pain to the patient and could result in a sudden movement resulting in further injury or failed actuation of the device. It will be understood by a person skilled in the art that the sliding component 1119 shown in FIG. 9 can be used with any of the injection devices described above with reference to FIGS. 1 to 8.

In another embodiment (not shown), the sliding component comprises a flange which extends inwardly and outwardly from an end of the sliding component.

A further embodiment of a sliding component 2119 is shown in FIG. 10. In this embodiment, a first end of the sliding component 2119 flares 2119b (i.e. tapers) outwardly towards a contact surface 2119a. In the arrangement shown in FIG. 10, the sliding component 2119 flares 2119b uniformly outwardly towards the contact surface 2119a. In other words, the flare or taper 2119b is of a constant gradient. It will be understood, however, that this arrangement is merely exemplary, and other suitable flaring arrangements could equally be used. It will be understood by a person skilled in the art that the sliding component 2119 shown in FIG. 10 can be used with any of the injection devices described above with reference to FIGS. 1 to 8.

An advantage arising from a sliding component which flares outwardly towards a contact surface, such as a flange, at its first end, is that the risk of the skin of a patient being trapped between the flange and the housing when the sliding component is moved into the housing is significantly reduced. This is advantageous because the trapping of skin could cause pain to the patient and could result in a sudden movement resulting in further injury.

Any of the contact surfaces, such as flanges, described herein can be integral to the sliding component i.e. the flange and the sliding component form a single, integrated part.

Alternatively, any of the flanges described above can be removable from the sliding component. Moreover, the flanges can be retrofitted to the sliding component. In one embodiment, a single sliding component is designed so as to be compatible with one, a plurality of, or all of the exemplary flanges described herein, and the various flanges are removable and replaceable depending on the preference of a user. The flange(s) and the sliding component or the injection device as a whole can be presented as a kit for convenience of storage and transport.

In embodiments where the sliding component comprises a contact surface at its first end, it is beneficial for the cap to be shaped so as to shapingly conform with the contact surface when the cap is over the exit aperture. A cap 1111 according to an embodiment of the invention is shown in FIG. 11. The cap 1111 comprises a body 1002 and a sleeve 1004 located within the body 1002 and fixed relative to the body 1002. An interior of the cap 1111 is shaped to receive a contact surface of a sliding component by means of a plurality of ribs 1006. In such cases, the ribs 1006 permit the cap to shapingly conform with the contact surface when the cap 1111 is located on the first end of the injection device, over an exit aperture. This helps to protect the first end of the sliding component and, in particular, the contact surface so as to minimise the risk of damage or contamination to the device. In particular, the cap 1111 shown in FIG. 11 comprises ribs 1006 having a portion 1008 with a reduced diameter to accommodate the contact surface. It will be understood by a person skilled in the art that the cap 1111 shown in FIG. 11 can be used with any of the injection devices described above with reference to FIGS. 1 to 8.

It is also beneficial for the case nose 129 to be shaped to permit it to accommodate the contact surface when the sliding component is moved in a direction into the housing. For example, it can be advantageous for the length of the case nose 129 to be reduced compared to conventional devices to accommodate the contact surface. In one embodiment, the length of the case nose 129 is reduced by approximately 1.5 mm compared to conventional devices. Moreover, in some examples, the case nose 129 is flattened to accommodate the thickness of the contact surface.

In any of the embodiments described herein, the flange can have any suitable dimensions. In one embodiment, the flange is approximately 1 mm in thickness (i.e. in the direction of the longitudinal axis of the device).

In use, such an injection device as described above might be used to deliver substances such as: golimumab, hormones, antitoxins, substances for the control of pain, substances for the control of thrombosis, substances for the control or elimination of infection, peptides, proteins, human insulin or a human insulin analogue or derivative, polysaccharide, DNA, RNA, enzymes, antibodies, oligonucleotide, antiallergics, antihistamines, anti-inflammatories, corticosteroids, disease modifying anti-rheumatic drugs, erythropoietin, or vaccines, for use in the treatment or prevention of rheumatoid arthritis, psoriatic arthritis, ankylosing spondylitis, ulcerative colitis, hormone deficiency, toxicity, pain, thrombosis, infection, diabetes mellitus, diabetic retinopathy, acute coronary syndrome, angina, myocardial infarction, atherosclerosis, cancer, macular degeneration, allergy, hay fever, inflammation, anaemia, or myelodysplasia, or in the expression of protective immunity. In addition to these substances, any medicament contained within the injection device may also include other substances, such as inactive ingredients, as a skilled person would appreciate.

It will of course be understood by the person skilled in the art that particular substances are efficacious for use in the treatment or prevention of particular conditions, as is well known in the art. For instance, it is known that antiallergics are efficacious for use in the treatment or prevention of allergies; antihistamines are efficacious for use in the treatment or prevention of hay fever; anti-inflammatories are efficacious for use in the treatment or prevention of inflammation; and so on.

Accordingly, any selection of one or more substances listed herein or in the claims for use in the treatment or prevention of one or more conditions for which those substance(s) are known to be efficacious is envisaged.

In a particular example, however, golimumab is known to be efficacious for use in the treatment or prevention of one or more of rheumatoid arthritis, psoriatic arthritis, ankylosing spondylitis or ulcerative colitis, or any combination of rheumatoid arthritis, psoriatic arthritis, ankylosing spondylitis and ulcerative colitis, or all of rheumatoid arthritis, psoriatic arthritis, ankylosing spondylitis and ulcerative colitis.

Golimumab may optionally be used in combination with one or more inactive ingredients such as any or all of L-histidine, L-histidine monohydrochloride monohydrate, sorbitol, polysorbate 80, and water. Golimumab may present in a composition in which golimumab is the only active ingredient. For example, golimumab may administered as SIMPONI®.

It will of course be understood that the present invention has been described above purely by way of example and modifications of detail can be made within the scope of the invention.

1. An injection device comprising:
a housing adapted to receive a syringe having a discharge nozzle, the syringe being moveable in the housing on actuation of the injection device along a longitudinal axis from a retracted position in which the discharge nozzle is contained within the housing and an extended position in which the discharge nozzle of the syringe extends from the housing through an exit aperture;
a sliding component which protrudes, when in a first position, from the aperture in the housing,
wherein the sliding component is movable into the housing into a second position,
wherein the sliding component comprises a contact surface located at a first end of the sliding component which protrudes from the housing when the sliding component is in its first position.

2. A device according to claim 1, wherein the sliding component is a sliding sleeve.

3. A device according to claim 1 or claim 2, further comprising:
an actuator; and
a drive adapted to be acted upon by the actuator and in turn act upon the syringe to advance it from its retracted position to its extended position and discharge its contents through the discharge nozzle.

4. A device according to any one of the preceding claims, wherein the sliding component is part of a locking mechanism moveable from an engaged position in a direction into the housing at the exit aperture into a disengaged position and adapted to prevent actuation of the device when it is in its engaged position and permit actuation of the device when it is in its disengaged position.

5. A device according to any one of the preceding claims, wherein the contact surface comprises a flange.

6. A device according to any one of the preceding claims, wherein the contact surface extends perpendicularly to a longitudinal axis of the sliding component.

7. A device according to any one of the preceding claims, wherein the first end of the sliding component flares outwardly towards the contact surface.

8. A device according to claim 7, wherein the sliding component flares uniformly outwardly towards the contact surface.

9. A device according to any one of the preceding claims, wherein the contact surface extends outwardly from the sliding component.

10. A device according to any one of the preceding claims, wherein the contact surface is located on an exterior surface of the first end of the sliding component.

11. A device according to any one of the preceding claims, wherein the contact surface extends inwardly from the sliding component.

12. A device according to any one of the preceding claims, wherein the contact surface is located on an interior surface of the first end of the sliding component.

13. A device according to any one of the preceding claims further comprising a cap removably located over the exit aperture.

14. A device according to claim 13, wherein the cap comprises a body and a sleeve located within the body and fixed relative to the body.

15. A device according to claim 13 or claim 14, wherein an interior of the cap is shaped to receive the contact surface.

16. A device according to claim 15, wherein the interior of the cap comprises at least one rib.

17. A device according to claim 16, wherein the interior of the cap comprises a plurality of ribs.

18. A device according to claim 16 or claim 17, wherein the at least one rib permits the cap to shapingly conform with the contact surface when the cap is over the exit aperture.

19. A device according to any one of claims 16 to 18, further comprising a retaining means on the housing and/or the cap for retaining the cap over the exit aperture.

20. A device according to any one of the preceding claims wherein a first end of the housing closest to the contact surface is shaped to receive the contact surface when the sliding component is moved in a direction into the housing.

21. A device according to any one of the preceding claims, wherein the contact surface is removable from the sliding component.

22. A device substantially as hereinbefore described with reference to the accompanying drawings.

23. An injection device according to any of the preceding claims containing a substance selected from the group consisting of:
golimumab, hormones, antitoxins, substances for the control of pain, substances for the control of thrombosis, substances for the control or elimination of infection, peptides, proteins, human insulin or a human insulin analogue or derivative, polysaccharide, DNA, RNA, enzymes, antibodies, oligonucleotide, antiallergics, antihistamines, anti-inflammatories, corticosteroids, disease modifying anti-rheumatic drugs, erythropoietin, or vaccines,
for use in the treatment or prevention of rheumatoid arthritis, psoriatic arthritis, ankylosing spondylitis, ulcerative colitis, hormone deficiency, toxicity, pain, thrombosis, infection, diabetes mellitus, diabetic retinopathy, acute coronary syndrome, angina, myocardial infarction, atherosclerosis, cancer, macular degeneration, allergy, hay fever, inflammation, anaemia, or myelodysplasia, or in the expression of protective immunity.

24. A substance selected from the group consisting of: golimumab, hormones, antitoxins, substances for the control of pain, substances for the control of thrombosis, substances for the control or elimination of infection, peptides, proteins, human insulin or a human insulin analogue or derivative, polysaccharide, DNA, RNA, enzymes, antibodies, oligonucleotide, antiallergics, antihistamines, anti-inflammatories, corticosteroids, disease modifying anti-rheumatic drugs, erythropoietin, or vaccines,
for use in the treatment or prevention of rheumatoid arthritis, psoriatic arthritis, ankylosing spondylitis, ulcerative colitis, hormone deficiency, toxicity, pain, thrombosis, infection, diabetes mellitus, diabetic retinopathy, acute coronary syndrome, angina, myocardial infarction, atherosclerosis, cancer, macular degeneration, allergy, hay fever, inflammation, anaemia, or myelodysplasia, or in the expression of protective immunity,
by delivery of said substance to a human subject using an injection device according to any one of claims 1 to 22.

25. An injection device for use in the treatment or prevention rheumatoid arthritis, psoriatic arthritis, ankylosing spondylitis, ulcerative colitis, hormone deficiency, toxicity, pain, thrombosis, infection, diabetes mellitus, diabetic retinopathy, acute coronary syndrome, angina, myocardial infarction, atherosclerosis, cancer, macular degeneration, allergy, hay fever, inflammation, anaemia, or myelodysplasia, or in the expression of protective immunity,
by delivery of a substance selected from the group consisting of: golimumab, hormones, antitoxins, substances for the control of pain, substances for the control of thrombosis, substances for the control or elimination of infection, peptides, proteins, human insulin or a human insulin analogue or derivative, polysaccharide, DNA, RNA, enzymes, antibodies, oligonucleotide, antiallergics, antihistamines, anti-inflammatories, corticosteroids, disease modifying anti-rheumatic drugs, erythropoietin, or vaccines,
to a human subject by using the injection device, wherein the injection device is an injection device according to any one of claims 1 to 22.
